(19) United States
(12) Patent Application Publication
(10) Pub. No.: US 2023/0168238 A1
(43) Pub. Date: Jun. 1, 2023

(54) IMAGING PROCESS AND SYSTEM

(71) Applicant: MASTER DYNAMIC LIMITED, Hong Kong (CN)

(72) Inventors: Yau Chuen YIU, Hong Kong (CN); Koon Chung HUI, Hong Kong (CN); Yan Yan MOK, Hong Kong (CN)

(73) Assignee: MASTER DYNAMIC LIMITED, Hong Kong (CN)

(21) Appl. No.: 17/919,406

(22) PCT Filed: Apr. 16, 2021
(86) PCT No.: PCT/CN2021/087858

§ 371 Date: Oct. 17, 2022

(30) Foreign Application Priority Data
Apr. 17, 2020 (HK) 32020006075.6

(51) Int. Cl. G01N 33/38; G01N 23/046; G01N 23/20025

(52) U.S. Cl. CPC G01N 33/381; G01N 23/046; G01N 23/20025

(57) ABSTRACT

A system (300) for providing a three-dimensional computer tomography image of a gemstone, the system (300) comprising an X-ray source (330) for providing an X-ray towards a gemstone (320); an X-ray detector system for detecting X-rays transmitted through or diffracted by the gemstone (320). The X-ray detector system surrounds the gemstone (320) and detects a three-dimensional multi-angle X-ray diffraction pattern from the gemstone (320) upon rotation of the gemstone (320) within the X-ray field, and provides an output signal therefrom, wherein the output signal provides for invasive three-dimension multiangle X-ray diffraction reconstructed computed tomography from the three-dimension multiangle X-ray diffraction pattern.

### TECHNICAL FIELD

The present invention relates to tomography imaging process and system. More particularly, the present invention provides a Tomography Imaging process and system for providing crystallographic information of a gemstone and in particular a diamond.

### BACKGROUND OF THE INVENTION

As is known, diamonds are a key component utilized in many luxury goods and items, in particular in articles of jewellery, and diamonds can have a very significant value.

As is known, the value of a diamond can depend on several physical properties of the diamond, and there are four main globally accepted standards utilised to assess the quality of a diamond, typically known in the gemstone industry as the 4C's, which correspond to the gemstone properties of clarity, colour, cut and carat weight.

By way of example, the Gemological Institute of America (GIA) has a clarity grade as shown below:

GIA CLARITY SCALE

| | | | | | | | | | | |
|---|---|---|---|---|---|---|---|---|---|---|
| FLAWLESS | INTERNALLY | VVS₁ | VVS₂ | VS₁ | VS₂ | SI₁ | SI₂ | I₁ | I₂ | I₃ |
| | FLAWLESS | | | | | | | | | |

| | | | |
|---|---|---|---|
| VERY VERY | VERY SLIGHTLY | SLIGHTLY | INCLUDED |
| SLIGHTLY | INCLUDED | INCLUDED | |
| INCLUDED | | | |

For the assessment on the clarity of a diamond, the assessment is based upon the quantity, size, and position of defects within the stone are required to be determined.

Inside the body of diamond, there may exist impurities, voids and cracks, which are considered internal defects.

At the diamond surface, there can be under-polished irregularities and scratches, which may be considered external defects.

These internal and external characteristics of a diamond are also important with respect to a diamond, as they can be one of the unique identifying marks or “birthmarks” that can be used for the identification of a diamond.

Currently, the most accepted practice to determine a diamond's clarity is by trained gemologist using a 10× microscope. Gemologists are trained for several months using standard diamond samples with different type of defects, with a view that a stone when assessed by different gemologists should reproduce the same assessment result for the clarity grade.

However, as can be noted, even under standardised training and assessment procedures, the repeatability of clarity assessment cannot necessarily be guaranteed, because of the unavoidable issue subjective human judgement.

Assessment on the same diamond by a same person at different time, may also result different clarity grades being applied to the same diamond. Because of human's vision tiredness, different judgement on the same diamond may also be made before and after assessments on many different stones.

Therefore, even for trained and experienced professional gemologists, such gemologists still experience difficultly for providing repeatability and consistency of clarity assessment.

### OBJECT OF THE INVENTION

It is an object of the present invention to provide a tomography imaging process and system, in particular a system and a process for providing crystallographic information of a gemstone, in particular a diamond, which overcomes or at least partly ameliorates at least some deficiencies as associated with the prior art.

### SUMMARY OF THE INVENTION

In a first aspect, the present invention provides a system for providing a three-dimensional computer tomography image of a gemstone, said system comprising:
an X-ray source for providing an X-ray towards a gemstone;
an X-ray detector system for detecting X-rays transmitted through or diffracted by the gemstone;
wherein said X-ray detector system surrounds the gemstone and detects a three-dimensional multi-angle X-ray diffraction pattern from the gemstone upon rotation of the gemstone within the X-ray field, and
wherein said X-ray detector system provides an output signal therefrom, wherein said output signal provides for invasive three-dimension multiangle X-ray diffraction reconstructed computed tomography from the three-dimension multiangle X-ray diffraction pattern.

The instrument may built with X-ray conventional computed tomography machine.

The system may include a plurality X-ray detectors.

The system may further include a sample stage.

The sample stage may be a 3-axis linear and rotational stage.

The sample stage may be disposed at a cross point of all detectors and X-ray source.

The X-ray source may be common laboratory accessible source, a conically diverged wave, a spherical wave, or a collimated wave.

The X-ray detector system may comprise four orthogonally disposed X-ray detectors.

A system according to claim 11, wherein the four X-ray detectors are arranged to form a front and bottom-end opened cage surrounding the gemstone.

The multiangle X-ray diffraction pattern may be captured for each rotation angle of the gemstone.

The system preferably provides for a high spatially resolved sample plane orientation image which is reconstructable by the three-dimension multiangle X-ray diffraction pattern.

The gemstone is preferably a diamond.

A process for providing a three-dimensional computer tomography image of a gemstone, said process including the steps of:
(i) providing a system according to the first aspect,
(ii) rotating a gemstone within an X-ray emission field from said X-ray source;

wherein said X-ray detector system surounds the gemstone and detects a three-dimensional multi-angle X-ray diffraction pattern from the gemstone upon rotation of the gemstone within the X-ray field, and said X-ray detector system provides an output signal therefrom, wherein said output signal provides for invasive three-dimension multiangle X-ray diffraction reconstructed computed tomography from the three-dimension multiangle X-ray diffraction pattern.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1 shows a schematic diagram of the experimental setup of the Diffraction Contrast Tomography (DCT) with synchrotron X-ray source, for explanatory purposes;

FIG. 2 shows a schematic diagram of the experimental setup of the DCT in laboratory X-ray source, for explanatory purposes;

FIG. 3 shows a schematic diagram of an embodiment of an imaging system according to the present invention;

FIG. 4 shows an absorption image of diffraction spots from diamonds of a group of three diamonds contained within a container of charcoal powder, using a system and process according to the present invention;

FIG. 5a shows to a DCT image of a rough diamond using a system and process according to the present invention; and

FIG. 5b shows the appearance of the rough diamond as used for the generation of the DCT image as shown in Figure Sa.

### DETAILED DESCRIPTION OF THE DRAWINGS

The present inventors have identified shortcomings in the manner in which investigation of gemstones, in particular rough diamonds, and upon identification of the problems with the prior art, have provided a system and process which overcomes the problems of the prior art, and provides a system and process which is more consistent and reliable.

Crystallographic Imaging Techniques

Crystallographic information is important information pertaining to the properties of a material. Crystallographic information can demonstrate how the crystals are orientated in a material, and the strain within a material, for example.

Within the art, there exist techniques for retrieving crystallographic information, such as X-ray diffraction (XRD) and electron backscatter diffraction (EBSD). Although these methods are now accessible, the present inventors have identified certain limitations.

XRD is commonly used in laboratories as a technique for measuring crystallographic information of a material. An advantage of XRD is the non-destructive nature of X-ray, in particular to non-biological materials.

There have been some advances in measurement strategies, such as two-dimensional data collection [1]. XRD can also retrieve two-dimensional crystallographic information by sample mapping.

However, the applicable sample depth is limited by the attenuation of the X-ray beam in XRD, as such it has been identified by the present inventors that the applicability of XRD is also limited by the sample nature, geometry, thickness, surface flatness and the like.

Another technique, EBSD, can provide detailed crystallographic information of a material. Implemented in scanning electron microscope (SEM). EBSD detects electron backscattering pattern for the crystallographic information [2]. With electron scanning through the surface, two-dimensional crystallographic information may be retrieved from a material.

EBSD can be advanced into three-dimension (3D EBSD) by the combination of focused ion beam (FIB) [3]. 3D EBSD may be considered a standard of three-dimensional crystallography. However, as identified by the present inventors. 3D EBSD requires serial sectioning of a sample by FIB, which is a destructive testing method, prohibiting its application on time-evolution studies and precious samples such as gemstones including diamonds.

Developed from synchrotron, Diffraction Contrast Tomography (DCT) [4] [5] now can provide an alternative way to existing techniques to retrieve three-dimensional crystallographic information non-destructively.

Principle of Diffraction Contrast Tomography (DCT)

DCT is a technique combining X-ray computed tomography (CT) and XRD [6] [7]. CT is a well-developed technology in both medical and material sciences.

By using penetrating X-ray irradiation, the structural information of a material can be retrieved from absorption contrast [8]. This is of particular importance in non-destructive testing of materials.

DCT can retrieve crystallographic information such XRD and determine a microstructure non-destructively like CT can provide [4] [5].

The fundamental equation of DCT is the same as XRD, which is the Bragg's law [1][9]

2d sin θ=nλ

### Where:

λ is the wavelength.

d is the distance between each adjacent crystal planes,

θ is the Bragg angle at which one observes a diffraction peak, and

n is the order of reflection.

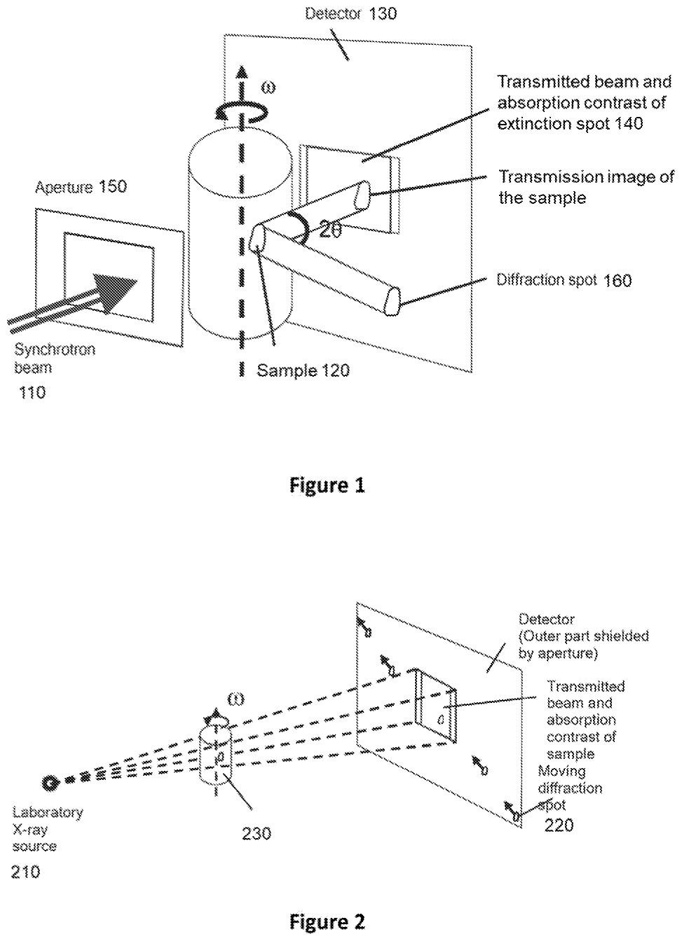

The experimental setup of DCT is similar to CT, and referring to FIG. 1 there is shown a schematic diagram of the experimental setup of the DCT with sychrontron X-ray source.

Synchrontron X-ray beam 110 is emitted by an X-ray source, and is irradiated on a sample 120 which is rotating between the X-ray source and X-ray detector 130.

When the sample 120 is irradiated by X-ray beam 110, photons interact with the material within the sample 120. Materials with higher density will give a lower transmission of X-ray 140 and vice versa. Therefore, when the transmitted X-ray beam 140 reach the detector 130, absorption contrast images can be formed.

At the same time, photons satisfying Bragg's law at the crystal grains will be diffracted away from the central main beam.

In order to capture these diffracted photons, an aperture 150 is provided to allow central X-ray beam illuminating the sample 120 only.

There will be no X-ray illuminating the outer part of the detector so the diffracted photons can be captured by the detector 130. These diffracted photons appear as diffraction spots 180. By conservation of energy, the crystalline grains satisfying Bragg's law will have less photons reaching the detector 130, thus leaving extinction spots with additional diffraction contrast.

The diffraction contrast of the extinction spots can give the structural information of the crystal grains satisfying the Bragg law. The position of the diffraction spots 180 can give the 2 θ information.

Hence, the orientations of the crystal grain can also be obtained. By correlating the diffraction spots and extinction spots, the three-dimensional structural and crystallographic information can be obtained non-destructively.

Development of the DCT Technology

DCT was first developed in synchrotron facilities, in which monochromatic X-ray beam is used.

More recently, the technology has been brought to laboratory for example (LabDCT) [10]. As in a synchrotron, the experimental setup is similar to CT with addition of aperture. The main difference is the nature of X-ray source.

FIG. 2 shows a schematic diagram of the experimental set up DCT in laboratory X-ray source.

In a laboratory, there is no well-collimated and monochromatized parallel X-ray beam. Instead, the traditional method, using accelerated electrons to hit metal target, is used to produce polychromatic X-ray cone beam as the laboratory X-ray source 210.

Because of the continuous energy spectrum of polychromatic X-ray of the laboratory X-ray source 210, LabDCT has diffraction spots 220 moving in a wide range of angles instead of particular angle during rotation of the sample 230.

As such, the dispersion of λ gives a wide range of θ in Bragg's law. It is noted, because of the energy dependence of intensity as a result of Bremsstrahlung and characteristic radiations, the intensities of diffraction spots also change with the Bragg angle θ.

LabDCT has now been implemented in commercial X-ray instrument [11] [12]. The instrument uses polychromatic X-ray cone beam with energy up to 160 keV and is able to obtain three-dimensional crystallographic information over sample with volumes up to 8 mm3 [12].

Moreover, because of the non-destructive nature of LabDCT, the instrument is able to conduct time-evolving “4D” experiments, such as material change under heat or stress over time.

Present Invention—Application to Diamond Imaging

The present inventors have found that such DCT imaging technique can be applied in diamond imaging, to retrieve the crystallographic information within a sample diamond.

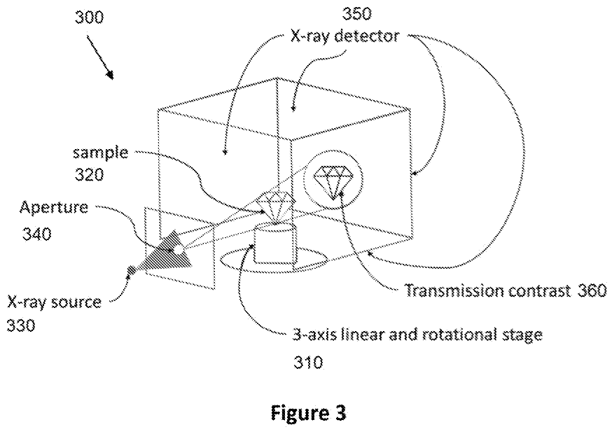

Referring now to FIG. 3, there is shown a schematic diagram of an embodiment of an imaging system 300 according to the present invention.

The diamond imaging system 300 comprises of a sample stage 310 for a sample diamond 320 to be placed on. The sample stage 310 is 3-axis linear movable and is rotatable about its central axis such that the sample diamond 320 can move and rotate upon irradiating by the X-ray beam emitted by the X-ray source 330.

This movable and rotatable sample stage 310 allows the entire surface of the diamond sample 320 placed thereon to be irradiated by the X-ray beam, and therefore providing a thorough examination to the crystallographic properties of the sample diamond 320.

In an embodiment of the present invention, the X-ray beam emitted by the X-ray source 330 is a conically diverged wave. Alternatively, the X-ray beam can also be a spherical wave or a collimated wave.

When the emitted X-ray photons reach the diamond sample 320, photons of the X-ray interact with the material within the diamond sample 320. Materials with higher density give a lower transmission of X-ray and vice versa. Therefore, when the transmitted X-ray beam reach the detector 350, transmission contrast images 360 can be formed.

At the same time, X-ray photons satisfying Bragg's Law at the crystal grains are diffracted away from the central main beam.

In order to capture these diffracted photons, an aperture 340 is provided to allow central X-ray beam illuminating the sample 320 only.

X-ray detectors 350 are arranged to detect the photons which are diffracted away from the central beam according to Bragg Law.

Diffraction of X-ray photons does not only occur along the direction of the incident X-ray but at all direction from the sample diamond 320. In order to collect more information signal regarding the diffracted photons from the sample diamond 320, multiple detectors are arranged, preferably surrounding the sample diamond 320 like a cage, such that more information can be captured.

There will be no X-ray illuminating the outer part of the detector so the diffracted photons can be captured by the detectors 350. These diffracted photons appear as diffraction spots. By conservation of energy, the crystalline grains satisfying the Bragg law will have less photons reaching the detector 350, thus leaving extinction spots with additional diffraction contrast.

The diffraction contrast of the extinction spots can give the structural information of the crystal grains satisfying Bragg's law. The position of the diffraction spots can give the 2 θ information.

Hence, the orientations of the crystal grain can also be obtained. By correlating the diffraction spots and extinction spots, the three-dimensional structural and crystallographic information can be obtained non-destructively.

Information received at each of the detectors 350 are then be added up to provide a more effective calculation and analysis to the crystallographic properties within the sample diamond 320.

Diamond is the single crystal of carbon with face-centered cubic (fcc) structure. The lattice constant is around 3.587 Å at 300 K [13]. Therefore, it is found that diamond can give sharp diffraction spots when illuminated with X-ray.

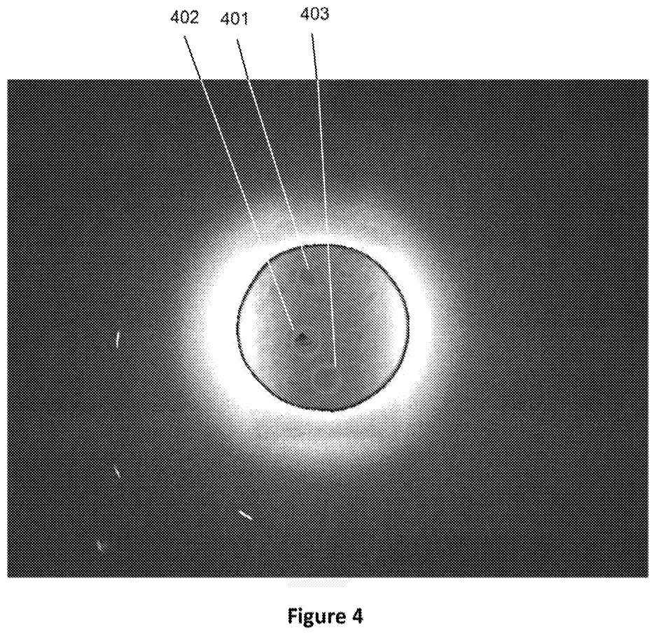

FIG. 4 shows the diffraction spots of 3 diamonds contained in a bottle of charcoal, an amorphous form of carbon using a system and process according to the present invention.

The three circles 401, 402 and 403 drawn indicate the position of the diamonds on the absorption contrast image inside the aperture. One of the diamonds, as indicated by circle 402, was stained with silver paint for ease of recognition. Only diamond single crystals give distinct diffraction spots.

The different diffraction behaviors of diamond crystals and amorphous carbon has been found to provide a useful tool in inspecting rough diamonds. Rough diamond is the uncut and unpolished raw diamond mined directly from the ore.

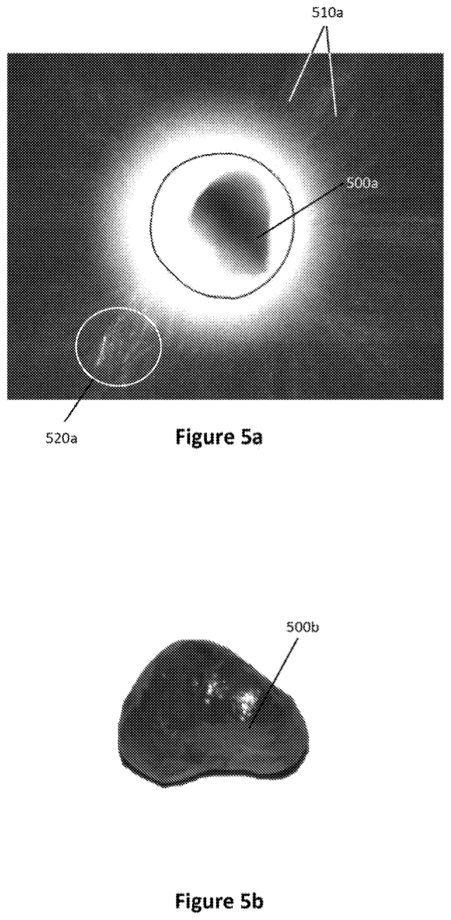

Rough diamonds 500b are not transparent on the surface and look dull as shown in FIG. 5b. Therefore, whether a rough diamond contains single crystal diamond inside is a question which cannot be readily answered.

Traditional X-ray CT can give a distribution of impurity of the rough diamond, however with no information regarding crystallinity. DCT can detect the presence of single crystals by sharp diffraction spots. This can assist the diamond industry to select high quality rough diamonds.

FIG. 5a is the DCT image of a rough diamond 500a, using a system and process according to the present invention. Only very fine and random distribution of diffraction spots 510a can be seen. This indicates that the rough diamond 500a contains only polycrystalline diamond.

The brighter rod shape spot 520a at the lower left corner indicates that there is a small region with better crystallinity but still not a sharp diffraction spot indicating the presence of a single crystal.

On the other hand, although polished diamond is the single crystal of carbon, most of the diamond is not perfect. These imperfections, if can be visible under 10× microscope, are called inclusions within the art.

Some of the inclusions are related to imperfection of crystallinity. For example, tiny crystals can be present inside the diamond, resulting in pin points, needles or clouds.

There can also be twinning, distortion or irregularities of crystal growth, which can result in grain center, internal graining or twinning wisp. [14] All these kinds of inclusions are crystal related so they can also be detected by DCT in principle.

### REFERENCES

[1] B. B. He, Two-Dimensional X-ray Diffraction, John Wiley & Sons, Inc., 2009.
[2] J. A. Venables and C. J. Harland, “Electron back-scattering patterns-A new technique for obtaining crystallographic information in the scanning electron microscope,” The Philosophical Magazine: A Journal of Theoretical Experimental and Applied Physics, vol. 27, no. 5, pp. 1193-1200, 1973.
[3] D. J. Rowenhorst, A. Gupta, C. R. Feng and G. Spanos, “3D Crystallographic and morphological analysis of coarse martensite: Combining EBSD and serial sectioning,” Scripta Materialia, vol. 55, no. 1, pp. 11-16, 2006.
[4] W. Ludwig, S. Schmidt, E. M. Lauridsen and H. F. Poulsen, “X-ray diffraction contrast tomography: a novel technique for three-dimensional grain mapping of polycrystals. I. Direct beam case,” Journal of Applied Crystallography, vol. 41, pp. 302-309, 2008.
[5] G. Johnson, A. King, M. G. Honnicke, J. Marrow and W. Ludwig, “X-ray diffraction contrast tomography: a novel technique for three-dimensional grain mapping of polycrystals. II. The combined case, Journal of Applied Crystallography, vol. 41, pp. 310-318, 2008.
[6] S. A. McDonald, P. Reischig, C. Holzner, E. M. Lauridsen. P. J. Withers, A. P. Merkle and M. Feser, “Non-destructive mapping of grain orientations in 3D by laboratory X-ray microscopy,” Scientific Reports, vol. 5. no. 14665. 2015.
[7] J. Banhart, “Three-Dimensional X-ray Diffraction,” in Advanced Tomographic Methods in Materials Research and Engineering, Oxford University Press, 2008. p. 249.
[8] S. R. Stock, MicroComputed Tomography: Methodology and Applications. CRC Press. 2009.
[9] C. Kittel, Introduction to Solid State Physics, John Wiley & Sons, Inc., 1996.
[10] A. King, P. Reischig. J. Adrien and W. Ludwig, “First laboratory X-ray diffraction contrast tomography for grain mapping of polycrystals,” Applied Crystallography, vol. 46, pp. 1734-1740, 2013.
[11] C. Holzner, L. Lavery, H. Bale, A. Merkle. S. McDonald, P. Withers, Y. Zhang, D. Juul Jensen, M. Kimura, A. Lyckegaard, P. Reischig and E. M. Lauridsen, “Diffraction Contrast Tomography in the Laboratory—Applications and Future Directions,” Microscopy Today, vol. 24. no. 4, pp. 34-42, 2016.
[12] L. Lavery, N. Gueninchault, H. Bale, C. Holzner, F. Bachmann and E. Lauridsen, “3D Mapping Grain Morphology and Grain Orientations by Laboratory Diffraction Contrast Tomography,” Microscopy and Microanalysis, vol. 23, no. S1, pp. 48-49, 2017.
[13] T. Sato, K. Ohashi, T. Sudoh, K. Haruna and H. Maeta, “Thermal expansion of a high purity synthetic diamond single crystal at low temperatures,” Physical Review B, vol. 85, p. 092102, 2002.
[14] “Diamond Inclusions Defined,” Gemological Institute of America, [Online]. Available: http://4cs.gia.edu/en-usiblog/diamond-indusions-defined/. [Accessed 19 Apr. 2018].

1. A system for providing a three-dimensional computer tomography image of a gemstone, said system comprising:
an X-ray source for providing an X-ray towards a gemstone;
an X-ray detector system for detecting X-rays transmitted through or diffracted by the gemstone;
wherein said X-ray detector system surrounds the gemstone and detects a three-dimensional multi-angle X-ray diffraction pattern from the gemstone upon rotation of the gemstone within the X-ray field, and
wherein said X-ray detector system provides an output signal therefrom, wherein said output signal provides for invasive three-dimension multiangle X-ray diffraction reconstructed computed tomography from the three-dimension multiangle X-ray diffraction pattern.

2. A system according to claim 1, wherein the system includes an X-ray conventional computed tomography machine.

3. A system according to claim 1, wherein the system includes a plurality of X-ray detectors.

4. A system according to claim 1, further comprising a sample stage.

5. A system according to claim 4, wherein the sample stage is a 3-axis linear and rotational stage.

6. A system according to claim 4, wherein the sample stage is disposed at a cross point of all detectors and X-ray source.

7. A system according to claim 1, wherein the X-ray source is a standard laboratory accessible source.

8. A system according to claim 1, wherein the X-ray source is a conically diverged wave.

9. A system according to claim 1, wherein the X-ray source is a spherical wave.

10. A system according to claim 1, wherein the X-ray source is a collimated wave.

11. A system according to claim 1, wherein the X-ray detector system comprises four orthogonally disposed X-ray detectors.

12. A system according to claim 11, wherein the four X-ray detectors are arranged to form a front and bottom-end opened cage surrounding the gemstone.

13. A system according to claim 1, wherein the three-dimension multiangle X-ray diffraction pattern is captured for each rotation angle of the gemstone.

14. A system according to claim 1, wherein said system provides a high spatially resolved sample plane orientation image which is re-constructable by a three-dimension multiangle X-ray diffraction pattern.

15. A system according to claim 1, wherein the gemstone is a diamond.

16. A process for providing a three-dimensional computer tomography image of a gemstone, said process including the steps of:
(i) providing a system according to claim 1,
(ii) rotating a gemstone within an X-ray emission field from said X-ray source;
wherein said X-ray detector system surrounds the gemstone and detects a three-dimensional multi-angle X-ray diffraction pattern from the gemstone upon rotation of the gemstone within the X-ray field, and
provides an output signal therefrom, wherein said output signal provides for invasive three-dimension multiangle X-ray diffraction reconstructed computed tomography from the three-dimension multiangle X-ray diffraction pattern.

17. A process according to claim 16, wherein the gemstone is a diamond.
